SEQUENCE LISTING (7 sequences)

SEQ ID NO: 1 - DNA, 372 bp, Artificial
  CTB nucleotide sequence
atgatcaagctcaagtttggagtgttcttcactgtgctccttagctctgcctatgcacatggcaccccacaaaacatcactgacttgtgtgctgagtaccacaacacccaaatccacaccctcaatgacaagatctttagctacaccgagagccttgctggcaagagggagatggctatcatcaccttcaagaatggtgctaccttccaagtggaggtgcctggaagccaacacattgatagccaaaagaaggccattgagaggatgaaggacacacttaggatagcttacctcactgaggctaaggtggagaagctttgtgtgtggaacaacaagaccccccatgctattgctgccatcagcatggccaac

SEQ ID NO: 2 - protein, 115 aa, Artificial
  CTB amino acid sequence
MGGLVVPHTKLLHLSLSEVSYPKCVLHPLNGLLLAINVLASRHLHLEGSTILEGDDSHLPLASKALGVAKDLVIEGVDLGVVVLSTQVSDVLWGAMCIGRAKEHSEEHSKLELDH

SEQ ID NO: 3 - DNA, 637 bp, Artificial
  MERS-CoV 377-588 nucleotides
acaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcggcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaataagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagctt

SEQ ID NO: 4 - protein, 137 aa, Artificial
  MERS-CoV 377-588 amino acids
MKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 5 - DNA, 843 bp, Artificial
  Fc nucleotide sequence
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaaggcctccagttaccaactcctgtctggtttcatgtccttttctatctggcagtgggaataatgtttttagtgaacactgttctctgggtgacaatacgtaaagaactgaaaagaaagaaaaagtggaatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtaa

SEQ ID NO: 6 - protein, 280 aa, Artificial
  Fc amino acid sequence
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFMEGEPLALRCHAWKDKLVYNVLYYRNGKAFKFFHWNSNLTILKTNISHNGTYHCSGMGKHRYTSAGISQYTVKGLQLPTPVWFHVLFYLAVGIMFLVNTVLWVTIRKELKRKKKWNLEISLDSGHEKKVISSLQEDRHLEEELKCQEQKEEQLQEGVHRKEPQGAT

SEQ ID NO: 7 - DNA, 1872 bp, Artificial
  Codon optimized sequence (CTB-S377-588-Fc)
atgattaagctcaagttcggtgttttctttacagtgctcctctcctccgcatacgctcacggtactccacaaaatataactgatctctgcgctgagtatcataatactcaaatccatacacttaacgataagattttctcttacactgagtcattggctggaaaaagggaaatggctattattacttttaagaatggtgctacatttcaagttgaggttcctggatcacaacatatcgattctcaaaagaaagctatcgaaaggatgaaggatactcttaggattgcttatttgacagaggctaaggttgaaaagctttgtgtttggaataacaagacacctcatgctattgctgctatttctatggctaatcaagctgagggtgttgaatgcgatttttctccacttttgtcaggaactccacctcaagtttacaacttcaagagattggtttttactaattgtaattacaatcttacaaagcttttgtcattgttttctgttaacgatttcacatgttcacaaatttctccagctgctatcgcttcaaactgctactcttcacttatcttggattacttctcttaccctctttctatgaagtctgatttgtcagtttcttcagctggtccaatctcacaattcaattacaagcaatcattttctaacccaacttgtcttatcttggctacagttcctcataatcttactacaatcacaaagccattgaagtactcttatattaataagtgttctagacttttgtcagatgataggactgaggttcctcaacttgttaacgctaaccaatactctccatgcgtttcaattgttccttctactgtttgggaggatggagattattacaggaagcaactttctccattggaaggaggtggatggcttgttgcttcaggatctactgttgctatgacagaacaattgcaaatgggttttggaattacagttcaatacggtactgatacaaattcagtttgccctaaactttggtttttgactacacttttgctttgggttccagttgatggacaagttgatactacaaaggctgttattactcttcaacctccttgggtttctgtttttcaagaagagactgttacattgcattgtgaagttcttcatttgcctggttcttcatctacacaatggtttttgaatggtactgctacacaaacttctacaccatcatacagaatcacttcagcttctgttaacgattcaggagagtacagatgccaaaggggtctttctggaagatcagatcctattcaattggaaattcataggggttggttgcttttgcaagtttcatctagagtttttatggagggagaaccacttgctttgaggtgtcatgcttggaaggataagcttgtttacaacgttttgtactacagaaatggtaaagcttttaagtttttccattggaactctaatcttactatcttgaagacaaacatctcacataatggtacttaccattgctctggtatgggaaaacataggtatacttcagctggtatttctcaatacacagttaagggacttcaattgccaactcctgtttggtttcatgttttgttttatcttgctgttggaattatgtttcttgttaacactgttttgtgggttacaatcagaaaggagcttaagaggaagaaaaagtggaatcttgagatttctttggattcaggtcatgaaaagaaggttatttcatctcttcaagaagatagacatcttgaagaggaattgaaatgtcaagagcaaaaggaggaacaattgcaagaaggagttcatagaaaggagcctcagggtgctacattagaagtgttgttccaaggaccataa

(19) United States
(12) Patent Application Publication
(10) Pub. No.: US 2020/0172920 A1
(43) Pub. Date: Jun. 4, 2020

(54) APPLICATION OF PLANT AS HOST IN EXPRESSING VACCINE OF MIDDLE EAST RESPIRATORY SYNDROME

(71) Applicant: Kevin WANG, West Linn, OR (US)

(72) Inventors: Kevin WANG, West Linn, OR (US); Wen LI, Wuhan, Hubei (CN); Shunchang JIAO, Beijing (CN); Weibin ZHOU, Seattle, WA (US); Shunxue TANG, Camarillo, CA (US)

(73) Assignee: Kevin WANG, West Linn, OR (US)

(21) Appl. No.: 16/621,853

(22) PCT Filed: Jul. 19, 2017
(86) PCT No.: PCT/CN2017/093498

§ 371 Date: Dec. 12, 2019

(30) Foreign Application Priority Data
Jun. 16, 2017 (CN) 201710458321.X

(51) Int. Cl. C12N 15/82; C07K 14/005

(52) U.S. Cl. CPC C12N 15/8258; C12N 15/8205; C07K 14/005; C12N 2770/20022; C07K 2319/30; C07K 2319/55; C12N 2770/20034; C12N 2800/22

(57) ABSTRACT

Provided is an application of a plant as a host in expressing a vaccine of Middle East respiratory syndrome. In particular, lettuce is utilized to transiently express fusion proteins to prepare a vaccine for Middle East respiratory syndrome.

The present application claims priority of Chinese Patent Application No. 201710458321.X, entitled “APPLICATION OF PLANT AS HOST IN EXPRESSING VACCINE OF MIDDLE EAST RESPIRATORY SYNDROME”, filed on Jun. 16, 2017 at the Chinese Patent Office, and the entire contents of which are incorporated herein by reference.

### FIELD OF THE INVENTION

The present invention relates to the field of biotechnology, and in particular to use of a plant as a host for expressing a vaccine for Middle East respiratory syndrome.

### BACKGROUND OF THE INVENTION

In April 2012, Middle East Respiratory Syndrome (MERS) induced by a new type of coronavirus (MERS-CoV) emerged in Saudi Arabia. Severe acute respiratory diseases occurred in people infected with MERS-CoV, and the symptoms include high fever, cough, shortness of breath, as well as some reported pneumonia and gastrointestinal symptoms. The first large-scale outbreak outside the Arabian Peninsula occurred in South Korea in May 2015, when the virus spread between people who were in close contact with each other, causing public panic. As of May 13, 2017, MERS-CoV infected 1,952 patients, resulting in 693 deaths, with a high mortality rate of approximately 35%. More than 27 countries have reported MERS-CoV cases.

MERS-CoV poses a threat to the public. Although the virus has temporarily disappeared, it may cause a potential sudden epidemic. Of particular concern is that no effective drugs or vaccines are currently available. Therefore, there is an urgent need to develop preventive treatments such as vaccines. The spike glycoprotein (S) protruding from the MERS-CoV envelope plays an important role in the viral infection process. It recognizes and binds to the dipeptidyl peptidase 4 (DPP4; also known as CD26) receptor present on the surface of the host cell and then causes the virus to enter the cell. The MERS-CoVS spike glycoprotein and receptor binding domain (RBD) have been shown to induce neutralizing antibodies to MERS-CoV. Unfortunately, no production system is capable of rapidly producing therapeutic antibodies in sufficient quantities. Therefore, there is an urgent need for a vaccine against MER-CoV. The candidate vaccines based on the S protein and RBD have been studied and have shown promising as a therapeutic intervention for MERS-CoV infection.

The MERS-CoV full-length recombinant S glycoprotein has been expressed in chicken embryo fibroblasts (CEF) by the modified vaccinia virus Ankara (MVA), and the results show that virus-neutralizing antibodies are induced in vaccinated mice. The synthetic anti-DNA protein DNA vaccine has been reported to produce potent humoral immune responses in mice, camels and non-human primates. The RBD domain in the S protein specifically binds to the DPP4 receptor on the host cell membrane and has been reported to be a subunit vaccine against MERS-CoV infection. Genes encoding different RBD fragments are cloned, fused to the Fc fragment of human IgG, and expressed in human embryonic kidney cells (HEK-293). In the RBD fragment tested, the fragment from residues 377 to 588 and fused to Fc (S377-588-Fc) induced the highest titer of IgG antibodies in mice and showed the highest receptor (DPP4) binding affinity. It also induced higher levels of serum antibody neutralizing MERS-CoV in immunized mice and rabbits. Using mF59 adjuvant, a concentration as low as 1 μg of RBD (S377-588-Fc) subunit vaccine can elicit strong neutralizing antibodies against pseudotyped viruses and live MERS-CoV viruses in mice. Intranasal delivery of RBD-based subunit proteins results in a more potent systemic cellular immune response compared with subcutaneous vaccination and a significantly higher local mucosal immune response in the mouse lung. These studies indicate that RBD-based fragments are ideal candidates for the development of potentially potent vaccines against the MERS-CoV virus.

The current recombinant MERS-CoV RBD-based subunit vaccine is mainly produced in human embryonic kidney cells (HEK293T). However, current protein production from HEK293T cells is relatively low, only showing sufficient yield in laboratory tests. Therefore, there is an urgent need for a more efficient production system to rapidly produce vaccines and sufficient vaccine quantities to respond to any MERS-CoV outbreak.

Currently, animal cells are used to produce nasal spray vaccines for Middle East Respiratory Syndrome (MERS-CoV). However, the culture of animal cell requires expensive culture fluids, strict plant conditions, complicated operations, a period of at least two weeks, and extremely high costs due to the low production of animal cell. Sometimes the virus carried by animal cells can infect humans, resulting in low safety. Therefore, it is of great practical significance to provide a plant as a host for the expression of a vaccine for Middle East respiratory syndrome.

### SUMMARY OF THE INVENTION

Therefore, the present invention provides use of a plant as a host for expressing a vaccine for Middle East respiratory syndrome. The invention utilizes a lettuce system to transiently express a nasal spray vaccine in a short time (4d). Lettuce basically does not contain plant toxic substances, and contains less fiber, which is beneficial to downstream protein purification and greatly reduces production costs. The cultivation of lettuce is simple, simplifying the operation steps. Moreover, plant viruses do not infect humans, greatly increasing the safety.

In order to achieve the above object, the present invention provides the following technical solutions.

The present invention provides use of a plant as a host for expressing a vaccine for Middle East respiratory syndrome.

In some specific embodiments of the invention, the plant is selected from the group consisting of lettuce, tobacco, Chinese cabbage, rice, corn, soybean and wheat; and the organ of the plant is selected from the group consisting of a leaf, a seed, a rhizome and a whole plant.

The invention also provides an expression vector comprising CTB, RBD-Fc and a vector.

In some specific embodiments of the invention, the codons of CTB or RBD-Fc are plant-preferred codons; the codon-optimized sequence of CTB and RBD-Fc fusion protein is set forth in SEQ ID NO: 7.

In some specific embodiments of the invention, the nucleotide sequence of CTB is set forth in SEQ ID NO: 1, and the amino acid sequence of CTB is set forth in SEQ ID NO: 2.

The nucleotide sequence of RBD is set forth in SEQ ID No: 3; and the amino acid sequence of RBD is set forth in SEQ ID NO: 4.

The nucleotide sequence of the Fc is set forth in SEQ ID No: 5; and the amino acid sequence of the Fc is set forth in SEQ ID NO: 6.

In some specific embodiments of the invention, the vector is a binary plant vector.

In some specific embodiments of the invention, the method of constructing the expression vector comprises the steps of:

Step 1: linking CTB with RBD-Fc to obtain CTB-5377-588-Fc;

Step 2: optimizing the codons of CTB, RBD and Fc to plant-preferred codons respectively, and linking the optimized CTB with the optimized RBD-Fc to obtain a codon-optimized CTB-5377-588-Fc sequence;

Step 3: adding KpnI restriction site at the 5′ end, and SacI and Pad sites at the 3′ end of the codon-optimized CTB-5377-588-Fc sequence and CTB-5377-588-Fc, and then generating pWT-CTB-RBD-Fc vector and pOP-CTB-RBD-Fc vector by ThermoFisher respectively; and

Step 4: producing gene fragments of WT-MersCoV and OP-MersCoV using KpnI/SacI, respectively, and cloning WT-MersCoV and OP-MersCoV into the binary plant expression vector pCam35S to obtain transient expression vectors of p35S-WT-MersCoV and p35S-OP-MersCoV respectively.

Specifically, the method for constructing the expression vector provided in the present invention is as follows.

To improve the expression and translation of proteins in the lettuce system, CTB-5377-588-Fc is redesigned to preferentially match the codons found in plants. The cholera toxin B subunit (CTB) shows an ability to increase antigen uptake and effectively induce mucosal responses. To improve the immunogenicity of the intranasal vaccine, CTB (Genbank ID: AY475128.1) is linked with RBD (Genbank ID: KM027288.1)-Fc (Genbank ID: BC 156864.1). The codon-optimized CTB-5377-588-Fc is designed and synthesized by GeneArt™ GeneOptimizer™ (ThermoFisher).

KpnI restriction site is added at the 5′ end and the SacI and PacI sites are added at the 3′ end of CTB-5377-588-Fc and the optimized sequence thereof, and pWT-CTB-RBD-Fc and pOP-CTB-RBD-Fc vectors are generated by ThermoFisher. The gene fragments are generated by KpnI/SacI and cloned into the binary plant expression vector pCam35S to generate the transient expression vectors p35S-WT-MersCoV and p35S-OP-MersCoV, respectively, which are confirmed to be full size sequence by double enzyme digestion. These two plant expression constructs are transformed into Agrobacterium tumefaciens GV3101 by electroporation using Multiporator (Eppendorf, Hamburg, Germany), respectively. The resulting strains are spread evenly on selective LB plates containing kanamycin antibiotic (50 mg/L). After incubating for 2 days at 28° C. in the dark, single colonies are picked and inoculated into 0.5 LYEB (yeast extract broth, 5 g/L sucrose, 5 g/L tryptone, 6 g/L yeast extract, 0.24 g/LMgSO₄, pH 7.2) supplemented with antibiotic (50 mg/L kanamycin) liquid medium. The inoculated culture is incubated at 25 to 28° C. for 72 h in a shaker (220 rpm). The OD600 value is measured and adjusted to 3.5 to 4.5 by adding YEB medium. The culture medium is then collected and centrifuged (4,500 rpm) for 10 min. The agrobacterium cells are resuspended in a penetrating medium (10 mM MES, 10 mM MgSO4) and O.D.600 is adjusted to 0.5.

The invention also provides use of the expression vector for expressing a vaccine for Middle East respiratory syndrome.

In addition, the present invention also provides a method for expressing a vaccine for Middle East respiratory syndrome in a plant host by transforming the expression vector provided by the present invention into agrobacterium, performing agrobacterium-mediated vacuum infiltration on the plant tissue, and then extracting and isolating the protein to obtain a vaccine for Middle East respiratory syndrome.

In some specific embodiments of the invention, the agrobacterium-mediated vacuum infiltration comprises the steps of:

Step 1: vacuuming for 25˜45s;

Step 2: maintaining under a vacuum of −95 kPa for 30˜60s;

Step 3: releasing the pressure to allow the penetrating fluid to penetrate into the plant tissue; and

repeating the above steps 2 to 3 times, and then keeping for 4d in the dark.

In some specific embodiments of the invention, the agrobacterium is Agrobacterium tumefaciens GV3101.

Specifically, the agrobacterium-mediated vacuum infiltration is performed by adding the prepared agrobacterium culture suspension into a 2 L beaker and placing the beaker in a desiccator. 10% of the front end of the lettuce leaves are removed with a knife, and the rest of the plant is inverted (core up) and gently spun in the bacterial suspension, and the desiccator was then sealed. Vacuum was applied using a vacuum pump (Welch Vacuum, Niles, Ill., USA) for about 25 to 45 s until bubble formation in the leaf space and the penetrating fluid in the leaf tissue are observed. After keeping under the pressure for 30˜60s, the pressure is released quickly, allowing the penetrating fluid to penetrate into the space inside the tissue. This procedure is repeated 2 to 3 times until the penetrating fluid diffuses significantly in the lettuce tissue. The lettuce tissue is then gently removed from the penetrating fluid and rinse three times with distilled water and then transferred to a container covered with a plastic film. The treated samples are kept in the dark for 4 days.

After vacuum infiltration of agrobacterium, the recombinant protein is obtained by extracting and isolating. The lettuce sample subjected to agrobacterium vacuum infiltrated was stirred with a stirrer and homogenized at a high speed in the extraction buffer (100 mM KPi, pH 7.8; 5 mM EDTA; 10 mM β-mercaptoethanol) at 1:1 ratio for 1 to 2 minutes. The homogenate is adjusted to pH 8.0, filtered through gauze, and the filtrate is centrifuged at 10,000 g at 4° C. for 15 min to remove cell debris. The supernatant is collected, mixed with ammonium sulfate (50%), and incubated on ice for 60 min with shaking. The mixture was again separated by a centrifuge (10,000 g) at 4° C. for 15 min. The resulting supernatant is subjected to a second round of ammonium citrate (70%) precipitation, sit on ice for 60 min with shaking, and again centrifuged at 10,000 g at 4° C. for 15 min. Then, the supernatant is discarded, and the precipitated protein of the treated sample is dissolved in 5 mL of a buffer (20 mM KPi, pH 7.8; 2 mM EDTA; 10 mM (3-mercaptoethanol) and stored at 4° C. The purified protein is further purified by the purification of a His-tagged protein. Approximately 200 μL of protein extract is mixed with 1 mL of equilibrated Ni-NTA agarose (Qiagen) buffer A (50 mM NaH₂PO₄, 300 mM NaCl, pH 8.0), and shaken on a shaker at 4° C. for 1 h. The mixture is then added to a pre-equilibrated 1 mL-polypropylene column with 1 mL Buffer B (50 mM NaH₂PO₄, 300 mM NaCl, 5 mM imidazole, pH 8.0). Thereafter, the mixture is washed with 10 mL of Buffer A, followed by 5 mL of Wash Buffer B. Purified His-tagged protein is eluted with elution buffer (50 mM NaH₂PO₄, 300 mM NaCl, 1 M imidazole, pH 8.0). The protein concentration is determined by Bradford method and Bradford kit (Bio-rad) is used to quantify the purified recombinant protein.

The obtained recombinant protein is subjected to SDS-PAGE gel electrophoresis and Western Blot. The purified protein extracted from the lettuce subjected to agrobacterium vacuum infiltrated is collected, and 5 μL of sample is heat-denatured (95° C.). The sample is mixed with loading buffer (Biorad, Hercules, Calif., USA) and subjected to electrophoresed in 4 to 12% Bolt® Bis-Tris Plus SDS-gel (ThermoFisher Scientific, Waltham, Mass., USA). The gel is stained with Coomassie Blue G250 (Biorad) and photographed. For Western Blot of the recombinant protein, 10 μl of recombinant sample is separated in 10˜20% Bolt® Bis-Tris Plus polyacrylamide gel, and the proteins after electrophoresis is transferred to polyvinylidene fluoride (PVDF) film. The His-tagged protein is then detected using a HisProbe-HRP kit (ThermoFischer Scientific) according to the manufacturer's instructions. The membrane is incubated in SuperSignal® Working solution and then exposed to a CL-exposure film (ThermoFischer Scientific).

DPP4 is a functional receptor for MERS-CoV and is important in regulating immune responses. The present invention analyzes the binding affinity of MERS-CoVRBD to DPP4 by performing a co-immunoprecipitation assay. Recombinant human DPP4 (Sigma-Aldrich, St. Louis, Mo.) and recombinant MERS-CoV RBD are incubated together and then separated using SDS-PAGE to detect the size of the resulting complex.

The present invention utilizes lettuce system to transiently express a nasal spray vaccine for Middle East respiratory syndrome, which produces high levels of protein in a relatively short period of time (4d). This approach minimizes biosafety issues because processed lettuce tissue is usually developed in fully enclosed facilities or containers without biological pollution problems. Lettuce basically does not contain plant toxic substances, and contains less fiber, which is beneficial to downstream protein purification. The use of lettuce systems to produce nasal spray vaccines can significantly reduce cycle times and production costs.

The results of the present invention indicate that the lettuce system can be a more efficient expression platform, providing a means for rapid, transient expression of recombinant proteins. The agrobacterium vacuum infiltration for lettuce is simple, rapid, and can reduce necrosis and increase the production of recombinant protein. Lettuce can increase protein yield by withstanding vacuum pressure and allow for a more complete penetration of each leaf in the lettuce leaves. Since lettuce is easy to grow and commercially mass-produced, it is easier to obtain and cheaper than other transiently expressed plants, such as tobacco. This study used a stirrer to perform homogenization and demonstrated that the stirrer can be used for large-scale production of recombinant proteins because more lettuce tissue can be processed with a stirrer in a shorter period of time. Through moderate modification, the system can be used to produce functional recombinant proteins at high levels in a short period of time. In summary, the results of this invention provide a viable experimental basis for the industrialization of large-scale production of bioactive components of medicinal proteins in lettuce systems, and also provide vaccine for the future sudden infection of MERS-CoV.

### BRIEF DESCRIPTION OF THE FIGURES

In order to more clearly illustrate the embodiments of the present invention or the technical solutions in the prior art, the drawings to be used in the embodiments or the description of the prior art will be briefly described below.

FIG. 1 shows the constructs of Mers-CoV WT and optimized gene (constructed and synthesized by ThermoFisher).

FIG. 2 shows a diagram of the construction of the plant binary expression vector p355-OP-MersCoV and p35S-WT-MersCoV. WT-CTB-RBD-Fc and CTB-RBD-Fc fragments of FIG. 1 are generated by restriction endonuclease double digestion (KpnI/SacI), and then the fragments are ligated into the KpnI/SacI sites of pCam35S to generate plant binary expression vectors p35S-OP-MersCoV and p35S-WT-MersCoV.

FIG. 2 (A) shows p35S-WT-MersCoV and FIG. 2 (B) shows p35S-OP-MersCoV;

wherein, 35S is the CaMV35S promoter with tobacco mosaic virus (TMV) 5′UTR; NPTII is used for the expression of nptII gene for kanamycin resistance; Nos 3′ is a terminator; wild type and plant codon-optimized sequences; CTB is cholera toxin B subunit; RBD is MERS-CoV receptor binding domain (S377-588); Fc is Fc fragment of human IgG.

FIG. 2 (C) shows the enzyme digestion (KpnI/SacI) of p35S-OP-MersCoV and p35S-OP-MersCoV fragments; lane 1 shows WT-CTB-RBD-Fc fragment and lane 2 shows OP-CTB-RBD-Fc fragment.

FIG. 3 shows SDS-PAGE detection of the affinity reaction between purified CTB-RBD-Fc and DPP4. Lane 1: purified recombinant CTB-RBD-Fc (WT) (5 μg); Lane 2: purified recombinant CTB-RBD-Fc (OP) (5 μg); Lane 3: DPP4 (5 μg); Lane 4: DPP4+CTB-RBD-Fc (WT) (5 μg); Lane 5: DPP4+CTB-RBD-Fc (OP) (5 μg); Lane 6: sample without vacuum infiltration treatment as negative control.

### DETAILED DESCRIPTION OF THE INVENTION

The invention discloses use of a plant as a host for expressing a vaccine for Middle East respiratory syndrome. Those skilled in the art can learn from the contents of this document and appropriately improve the process parameters. It is specifically to be understood that all such alternatives and modifications are obvious to those skilled in the art and are considered to be included in the present invention. The methods and applications of the present invention have been described by the preferred embodiments, and it is obvious that the methods and applications described herein can be modified or appropriately changed and combined to implement and apply the techniques of the present invention without departing from the scope of the present invention.

The invention utilizes lettuce as an effective platform for recombinant protein production. The growth time of tobacco plants used for vacuum agrobacterium infiltration is usually 4 to 6 weeks. The invention eliminates the plant growth cycle and greatly saves the time for planting plants in the early stage. The exogenous MERS-CoV vaccine protein was expressed in a lettuce system and successfully isolated under mild conditions, demonstrating that the lettuce expression platform can be used to produce MERS-CoV vaccines against large-scale sudden infection of MERS-CoV.

The materials and reagents used in the application of the plant as a host for expressing a vaccine for Middle East respiratory syndrome according to the present invention are commercially available.

The present invention is further illustrated below in conjunction with the examples.

### Example 1. Construction of Plant Transient Expression Vector

To improve expression and translation of proteins in the lettuce system, in the present invention, the codons of CTB-S377-588-Fc were redesigned to preferentially match the codon frequency found in plants (sequences are set forth in SEQ ID NO: 7). The cholera toxin B subunit (CTB) is shown to increase antigen uptake and effectively induce mucosal responses. To improve the immunogenicity of the intranasal vaccine, CTB (Genbank ID: AY475128.1) was fused to RBD (Genbank ID: KM027288.1)-Fc (Genbank ID: BC156864.1). The optimized codons for CTB-5377-588-Fc were designed and synthesized by GeneArt™ GeneOptimizer™ (ThermoFisher).

KpnI restriction site was added at the 5′ end and the SacI and Pad sites were added at the 3′ end of CTB-5377-588-Fc and the optimized sequence thereof pWT-CTB-RBD-Fc and pOP-CTB-RBD-Fc vectors were generated by ThermoFisher. The gene fragments were obtained by KpnI/SacI digestion and cloned into the binary plant expression vector pCam35S to generate the transient expression vectors p35S-WT-MersCoV and p35S-OP-MersCoV, respectively, which were confirmed to be full size fragment by double enzyme digestion. There two plant expression constructs were transformed into Agrobacterium tumefaciens GV3101 by electroporation using Multiporator (Eppendorf, Hamburg, Germany), respectively. The resulting strains were spread evenly on selective LB plates containing kanamycin antibiotic (50 mg/L). After incubating for 2 days at 28° C. in the dark, single colonies were picked and inoculated into 0.5 LYEB (yeast extract broth, 5 g/L sucrose, 5 g/L tryptone, 6 g/L yeast extract, 0.24 g/LMgSO₄, pH 7.2) supplemented with antibiotic liquid medium (50 mg/L kanamycin). The inoculated culture was incubated at 25 to 28° C. for 72 h in a shaker (220 rpm). The OD 600 value was measured and adjusted to 3.5 to 4.5 by adding YEB medium. The culture medium was then collected and centrifuged (4,500 rpm) for 10 min. The agrobacterium cells were resuspended in penetrating medium (10 mM MES, 10 mM MgSO4) until O.D.600 was 0.5.

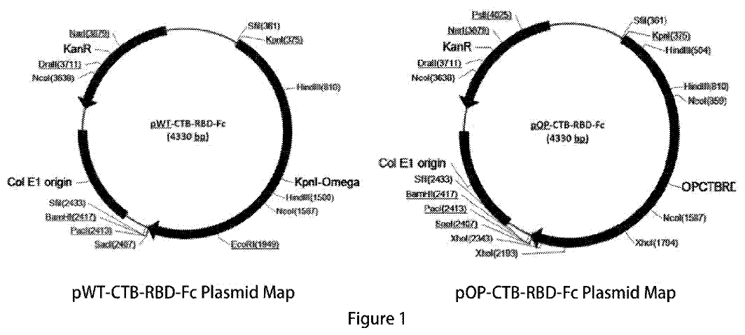

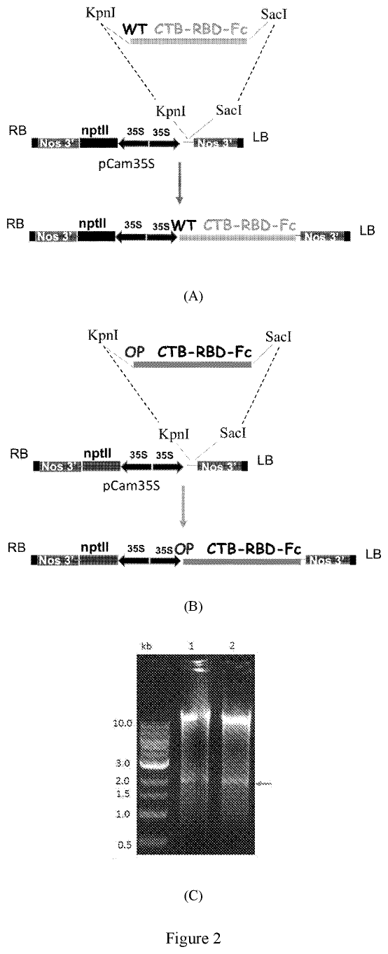

The gene fragments of WT-MersCoV and the optimized fragment OP-MersCoV are shown in FIG. 1. The two binary plant expression vectors p35S-WT-MersCoV and p35S-OP-MersCoV are constructed as shown in FIG. 2A, B. After completion of the construction, digestion was performed with specific restriction enzymes to confirm that the gene fragment was intact (FIG. 2C). Most of the lettuce tissue was submerged during vacuum infiltration. Except for the solid midrib area, the remaining parts turned to a yellowish brown area after 4 days of vacuum infiltration.

### Example 2. Agrobacterium-Mediated Vacuum Infiltration

The prepared agrobacterium culture suspension was added into a 2 L beaker and the beaker was placed in a desiccator. 10% of the top part of the lettuce leaves were removed with a knife, and the rest part was inverted (core up) and gently spun in the bacterial suspension, and the desiccator was then sealed. Vacuum was applied using a vacuum pump (Welch Vacuum, Niles, Ill., USA) for about 25 to 45 s until bubble formation in the leaf space and the penetrating fluid in the leaf tissue were observed. After keeping the lettuce under the pressure for 30-60s, the pressure was released quickly, allowing the penetrating fluid to penetrate into the space inside the tissue. This procedure was repeated 2 to 3 times until the penetrating fluid diffuses significantly in the lettuce tissue. The lettuce tissue was then gently removed from the penetrating fluid and rinsed three times with distilled water and then transferred to a container covered with a plastic film. The treated samples were kept in the dark for 4 days.

### Example 3. Protein Extraction and Isolation

The lettuce sample after agrobacterium vacuum infiltration was stirred with a stirrer and homogenized at a high speed in the extraction buffer (100 mM KPi, pH 7.8; 5 mM EDTA; 10 mM β-mercaptoethanol) at 1:1 ratio for 1 to 2 minutes. The homogenate was adjusted to pH 8.0, filtered through gauze, and the filtrate was centrifuged at 10,000 g at 4° C. for 15 min to remove cell debris. The supernatant was collected, mixed with ammonium sulfate (50%), and incubated on ice for 60 min with shaking, and then was again separated by a centrifuge (10,000 g) at 4° C. for 15 min. The resulting supernatant was subjected to a second round of ammonium citrate (70%) precipitation, sit on ice for 60 min with shaking, and again centrifuged at 10,000 g at 4° C. for 15 min. Then, the supernatant was discarded, and the precipitated protein of the treated sample was dissolved in 5 mL of a buffer (20 mM KPi, pH 7.8; 2 mM EDTA; 10 Mm β-mercaptoethanol) and stored at 4° C. The purified protein was further purified by the purification of the His-tagged protein. Approximately 200 μL of protein extract was mixed with 1 mL of equilibrated Ni-NTA agarose (Qiagen) buffer A (50 mM NaH₂PO₄, 300 mM NaCl, pH 8.0), and shaken on a shaker at 4° C. for 1 h. The mixture was then added to a pre-equilibrated 1 mL-polypropylene column with 1 mL Buffer B (50 mM NaH₂PO₄, 300 mM NaCl, 5 mM imidazole, pH 8.0). Thereafter, the mixture was washed with 10 mL of Buffer A, followed by 5 mL of Wash Buffer B to flow out by gravity. Purified His-tagged protein was eluted with elution buffer (50 mM NaH₂PO₄, 300 mM NaCl, 1 M imidazole, pH 8.0). The protein concentration was determined by Bradford method. Bradford kit (Bio-rad) was used to quantify the purified recombinant protein.

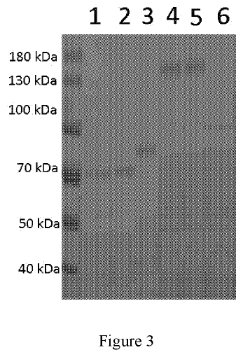

Downstream processing of plant-derived recombinant proteins is often difficult and expensive because cellulose cell walls are difficult to lyse and secondary plant metabolites are produced. In the present inventor, a stirrer is used to perform homogenization, which greatly saves the cost and simplifies the process of homogenization. The recombinant MersCoV vaccine protein was separated by SDS-PAGE and a band with an estimated molecular weight of approximately 70 kDa was observed in the lane (FIG. 3, lanes 1, 2), and there is no apparent corresponding bands in the negative control lane. The protein content of the purified sample was determined to be 0.58 mg based on the Bradford assay and the optical density measurement control group. In addition, a band of approximately 150 kDa was also detected in the affinity reaction between DPP4 and CTB-RBD-Fc (FIG. 3, lanes 4, 5), consistent with the predicted size.

### Example 4. SDS-PAGE Gel Electrophoresis and Western Blot

The purified protein extracted from the lettuce after agrobacterium vacuum infiltrated was collected. 5 μL sample was heat-denatured (95° C.), mixed with loading buffer (Biorad, Hercules, Calif., USA), and then subjected to electrophoresis on 4 to 12% Bolt® Bis-Tris Plus SDS-gel (ThermoFisher Scientific, Waltham, Mass., USA). After the completion of electrophoresis, the gel was stained with Coomassie Blue G250 (Biorad) and photographed again.

### Example 5. Binding of DPP4 to Recombinant MERS CoVRBD-Fc In Vitro

DPP4 is a functional receptor for MERS-CoV and is important in regulating immune responses. The binding affinity of MERS-CoVRBD to DPP4 was analyzed by performing co-immunoprecipitation assay. Recombinant human DPP4 (Sigma-Aldrich, St. Louis, Mo.) was incubated with recombinant MERS-CoV RBD. Samples were separated using SDS-PAGE to detect the size of the resulting complex.

When recombinant human DPP4 was incubated with recombinant MERS-CoVRBD, a band of approximately 150 kDa was isolated and detected by SDS-PAGE, demonstrating a significant affinity between recombinant MERS-CoV RBD and recombinant human DPP4.

### Example 6

Control group: a vaccine for Middle East respiratory syndrome generated in animals;

Experimental group 1: a vaccine for Middle East respiratory syndrome generated in the plant provided by the present invention;

Experimental group 2: a vaccine for Middle East respiratory syndrome generated in tobacco leaves.

TABLE 1
vaccines for Middle East respiratory syndrome

| | | | | | Production cost |
|---|---|---|---|---|---|
| | Production | Protein | | Difficulty in the downstream protein | (yuan per gram |
| Group | Time (d) | Concentration | | purification | of protein) |
| Control | 14 | 0.21 | mg/g | It is difficult to remove animal cell | 5000 |
| group | | | | impurities. In particular, animal cells | |
| | | | | are often contaminated with human | |
| | | | | viruses and have low safety. | |
| Experimental | 4**^{#} | 0.56**^{#} | mg/g | Relatively easy. The downstream | About 1200**^{##} |
| group 1 | | | | homogenization is carried out with a | yuan per gram of |
| | | | | stirrer, saving time and money, and | protein |
| | | | | eliminating the need to remove | |
| | | | | impurities such as nicotine and | |
| | | | | nicotine. | |
| Experimental | 7* | 0.32* | mg/g | Relatively difficult. The | About 1500* |
| group 1 | | | | time-consuming, laborious and | yuan per gram of |
| | | | | expensive liquid nitrogen grinding | protein |
| | | | | was required, and special steps were | |
| | | | | required to remove nicotine, | |
| | | | | nicotine impurities. | |

*indicates P ≤ 0.05 compared with the control group;
**indicates P ≤ 0.01 compared with the control group;
^{#}indicates P ≤ 0.05 compared with the experimental group 2;
^{##}indicates P ≤ 0.01 compared with the experimental group 2.

As can be seen from Table 1, compared with the animal system of the control group, the lettuce system provided by the present invention expresses the vaccine protein for Middle East respiratory syndrome, which reduces the production cycle very significantly (P≤0.01), improves the protein content very significantly (P≤0.01), simplifies the purification of the protein and reduces production costs very significantly (P≤0.01).

Compared with the tobacco leaf system of experimental group 2, the lettuce system expresses the vaccine protein for Middle East respiratory syndrome, which reduces the production cycle significantly (P≤0.05), improves the protein content significantly (P≤0.05), simplifies the purification of the protein and reduces production costs significantly (P≤0.01).

Compared with the animal system of the control group, the tobacco leaf system of experimental group 2 transiently expresses the vaccine protein for Middle East respiratory syndrome, which reduces the production cycle significantly (P≤0.05), improves the protein content significantly (P≤0.05), simplifies the purification of the protein and reduces production costs significantly (P≤0.05).

The above test results show that plant system, especially lettuce system, is a more economical and efficient expression platform for rapid and transient expression of recombinant proteins, which can produce large-scale vaccines of Middle East respiratory syndrome in a short period of time.

The above application of a plant as a host for expressing a vaccine for Middle East respiratory syndrome according to the present invention is described in detail. The principles and embodiments of the present invention are set forth herein in terms of specific examples, and the description of the above embodiments is only to aid in understanding the method of the present invention and its core concepts. It should be noted that those skilled in the art can make various modifications and changes to the present invention without departing from the spirit and scope of the invention.

1. A method for expressing a vaccine for Middle East respiratory syndrome, comprising using a plant as a host.

2. The method according to claim 1, wherein the plant is selected from the group consisting of lettuce, tobacco, Chinese cabbage, rice, corn, soybean and wheat; and the organ of the plant for use is selected from the group consisting of a leaf, a seed, a rhizome and a whole plant.

3. An expression vector comprising CTB, RBD-Fc and a vector.

4. The expression vector according to claim 3, wherein the codons of CTB and RBD-Fc are plant-preferred codons, and a codon-optimized sequence of CTB and RBD-Fc fusion protein is set forth in SEQ ID NO: 7.

5. The expression vector according to claim 3, wherein the vector is a binary plant vector.

6. The expression vector according to claim 3, wherein the method of constructing the expression vector comprises the steps of:
Step 1: linking CTB with RBD-Fc to obtain CTB-5377-588-Fc;
Step 2: optimizing the codons of CTB, RBD and Fc to plant-preferred codons respectively, and linking the optimized CTB with the optimized RBD-Fc to obtain a codon-optimized CTB-5377-588-Fc sequence;
Step 3: adding KpnI restriction site at the 5′ end, and SacI and Pad sites at the 3′ end of the codon-optimized CTB-5377-588-Fc sequence and CTB-5377-588-Fc sequence, and then generating pWT-CTB-RBD-Fc vector and pOP-CTB-RBD-Fc vector respectively; and
Step 4: producing gene fragments of WT-MersCoV and OP-MersCoV using KpnI/SacI, respectively, and cloning WT-MersCoV and OP-MersCoV into the binary plant expression vector pCam35S to obtain transient expression vectors of p35S-WT-MersCoV and p355-OP-MersCoV respectively.

7. A method for expressing a vaccine for Middle East respiratory syndrome, comprising using the expression vector according to claim 3.

8. The method according to claim 7, comprising
transforming the expression vector into agrobacterium,
performing agrobacterium-mediated vacuum infiltration on a plant tissue, and
extracting and isolating protein to obtain a vaccine for Middle East respiratory syndrome.

9. The method according to claim 8, wherein the agrobacterium-mediated vacuum infiltration comprises the steps of:
Step 1: vacuuming for 25˜45s;
Step 2: maintaining under a vacuum of −95 kPa for 30˜60s;
Step 3: releasing pressure to allow a penetrating fluid to penetrate into the plant tissue; and
repeating Step 1 to Step 3 for 2 to 3 times, and then keeping in dark for 4 days.
